(19) United States
(12) Patent Application Publication
(10) Pub. No.: US 2017/0333294 A1
(43) Pub. Date: Nov. 23, 2017

(54) Oral Examination Device

(71) Applicant: Christiannia Brown, Kingman, IN (US)

(72) Inventor: Christiannia Brown, Kingman, IN (US)

(21) Appl. No.: 15/597,507

(22) Filed: May 17, 2017

Related U.S. Application Data
(60) Provisional application No. 62/337,457, filed on May 17, 2016.

(51) Int. Cl. A61J 17/00; A61B 6/14; A61B 1/00; A61B 90/00; A61B 1/24; A61B 1/04; A61B 6/00; A61B 5/00

(52) U.S. Cl. CPC A61J 17/008; A61B 6/56; A61B 6/4057; A61B 6/145; A61B 5/0088; A61B 90/361; A61B 1/24; A61B 1/04; A61B 1/00016; A61B 5/0086; A61B 2090/3612

(57) ABSTRACT

An oral examination device including a lip shield, a nipple, and a camera. The lip shield defines a front side and back side. The nipple extends from the front side of lip shield. The nipple defines an interior cavity with an opening to the interior cavity provided from the rear of the lip shield. The camera is positioned in the interior cavity of the nipple.

### CROSS-REFERENCE TO RELATED APPLICATIONS

This application claims priority to U.S. provisional patent application Ser. No. 62/337,457, filed May 17, 2016, the entire contents of which are incorporated herein by reference.

### FIELD

This document relates to the fields of medical and dental examination, and particularly to devices for use in oral examinations.

### BACKGROUND

Medical examination and testing for certain individuals such as infants and toddlers is often difficult. This is particularly true when the area to be examined is in a location that is difficult for the medical professional to easily access and inspect. Examination of these areas often results in discomfort and trauma for the infant, toddler, or other patient during the examination and any related testing.

Oral examinations are one example of examination that occurs in a location that is difficult to access and inspect, particularly on an infant or toddler. In order to view the mouth, teeth and throat of the infant or toddler, the dental or other medical professional must convince the infant or toddler to open his or her mouth and leave the mouth open during the examination. In some instances, this may require some use of physical force to try to keep the infant or toddler's mouth open for the duration of the inspection. Forced opening of the mouth may further the discomfort and pain for the infant or toddler and significantly contribute to anxiety during the examination procedure.

In view of the foregoing, it would be advantageous to provide a device that allows for simplified and improved medical examinations and testing for infants, toddlers, and other individuals, and particularly simplified and improved oral examinations. It would also be advantageous if such a device allowed for more thorough medical and dental examinations and testing of the mouth, teeth and throat. Furthermore, it would also be advantageous if such device alleviated discomfort and trauma for the patient during oral examinations and other testing procedures, such as X-rays, therefore providing the medical or dental professional with better views of the patient's mouth, teeth and/or throat.

### SUMMARY

In accordance with one exemplary embodiment of the disclosure, there is provided an oral examination device including a lip shield, a nipple, and a camera. The lip shield defines a front side and back side. The nipple extends from the front side of lip shield. The nipple defines an interior cavity with an opening to the interior cavity provided from the rear of the lip shield. The camera is positioned in the interior cavity of the nipple.

Pursuant to another exemplary embodiment of the disclosure, there is provided a method of conducting an oral examination on a patient. The method includes inserting a nipple into a mouth of the patient, the nipple including an interior cavity with a camera positioned in the interior cavity. The method further includes obtaining images with the camera.

In accordance with yet another exemplary embodiment of the disclosure, there is provided an oral examination device including a flexible nipple designed and dimensioned to be received in the mouth of a patient, the flexible nipple defining an interior cavity. The oral examination device further includes a camera positioned in the interior cavity of the nipple.

The above described features and advantages, as well as others, will become more readily apparent to those of ordinary skill in the art by reference to the following detailed description and accompanying drawings. While it would be desirable to provide an oral examination device that provides one or more of any of the foregoing or other advantageous features, the teachings disclosed herein extend to those embodiments which fall within the scope of the appended claims, regardless of whether they accomplish one or more of the above-mentioned advantages.

### BRIEF DESCRIPTION OF THE DRAWINGS

FIG. 1 shows an exploded isometric view of a first exemplary embodiment of an oral examination device configured to obtain images of a mouth when inserted into the mouth.

FIG. 2 shows an opposite perspective view of the nipple and guard of the oral examination device of FIG. 1 including an opening to an interior cavity of the nipple.

FIG. 3 shows an exploded isometric view of a second exemplary embodiment of an oral examination device configured to obtain images of a mouth when inserted into the mouth.

FIG. 4 shows an exploded isometric view of a third exemplary embodiment of an oral examination device configured to obtain images of a mouth when inserted into the mouth.

### DESCRIPTION

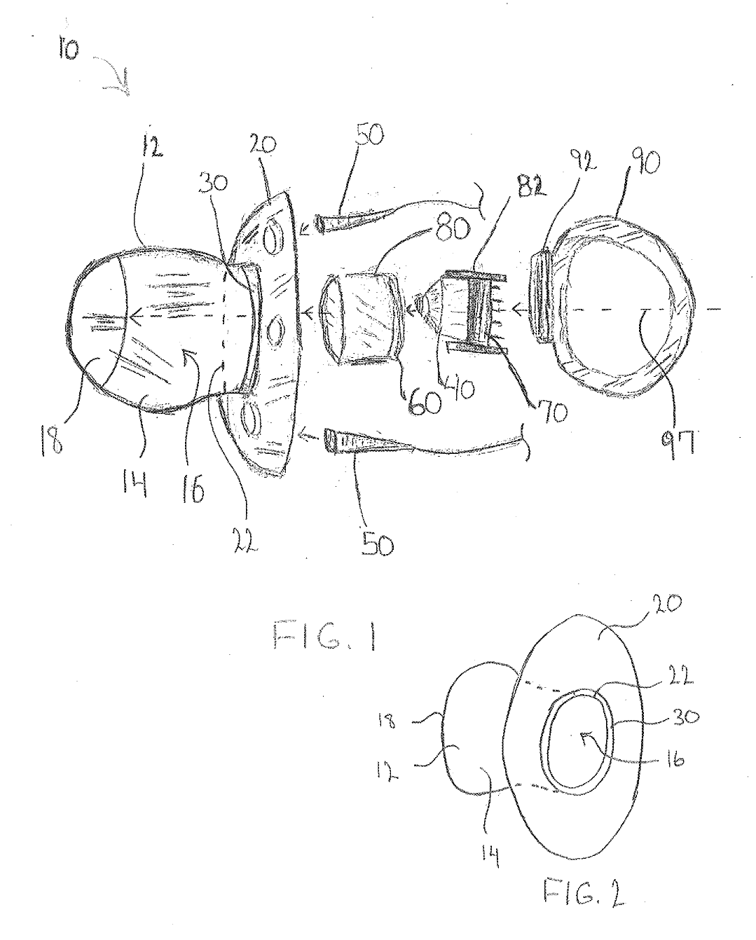

With reference to FIG. 1, in at least one embodiment an oral examination device 10 includes a nipple 12, a shield 20, a camera protector 30, a camera 40, a sensor 50, a sanitary gasket 60, a wireless connector 70, a basket 80, a handle 90 and clips 82. The nipple 12, shield 20 and handle 90 are arranged and provided in a configuration that is similar to that of a standard pacifier for use by infants and toddlers. FIG. 1 shows an exploded isometric view of the oral examination device. Accordingly, as explained in further detail below, it will be recognized that the camera protector 30, and camera 40 are both inserted into the nipple 12 when the oral examination device is assembled. The oral examination device 10 allows for simplified medical examinations and testing for infants and other individuals, and particularly simplified examinations of the mouth and throat.

The nipple 12 is configured in the form of a standard pacifier nipple configured to fit within the mouth of an infant. The nipple 12 includes an outer membrane 14 with a hollow interior that defines an interior cavity 16. It will be recognized that in at least one embodiment the nipple 12 is designed and dimensioned to fit properly within the mouth of the infant or toddler, allowing the infant to suck on the nipple 12 without extending too far into the mouth of the infant or toddler such that the nipple 12 chokes the infant or is uncomfortable to the infant. The outer membrane 14 that forms the nipple 12 is contoured in any of various shapes to conform to the interior of the mouth of a patient. For example, in at least one embodiment, a proximal base of nipple 12 near the shield 20 has a relatively small diameter, and a distal bulbous portion extending outward from the base, the bulbous portion defined by a diameter that is significantly greater than the diameter at the base. The outer membrane 14 is generally provided as monolithic construction and provided as a unitary piece with a congruent outer surface that is smooth to the touch (and the tongue and mouth of a patient).

In at least one embodiment, a portion of the outer membrane 14 is made of a substantially clear material (i.e., a transparent or translucent material) that allows a sufficient amount of light to pass there-through and allow for images to be seen through the outer membrane. In the embodiment of FIG. 1, at least a tip 18 of the nipple 12 provides a transparent area that that allows the camera 40 to obtain images through the transparent area. In other embodiments, the entire outer membrane may be comprised of a transparent material, or only limited portions may be comprised of a transparent material. For example, in various embodiments, a transparent area is only provided at the top of the nipple 12, or selected transparent areas associated with the teeth are provided along the sides of the nipple. Providing one or more selected transparent areas may assist the technician using the device in focusing on the proper portion of the mouth for the desired examination. Accordingly, various versions of the oral examination device 10 may be available for selection by a technician depending on the desired focus of the examination.

In at least one embodiment, the outer membrane 14 is comprised of a resilient flexible material, such as a thermoplastic made from any of various materials such as phthalates, BPA, lead, PVC-free silicone, or any of various other materials as will be recognized by those of skill in the art. Exemplary nipples 1 include those sold in association with various pacifiers such as the GumDrop® pacifier, Gerber® pacifier, or any of various other brands as will be recognized by those of ordinary skill in the art.

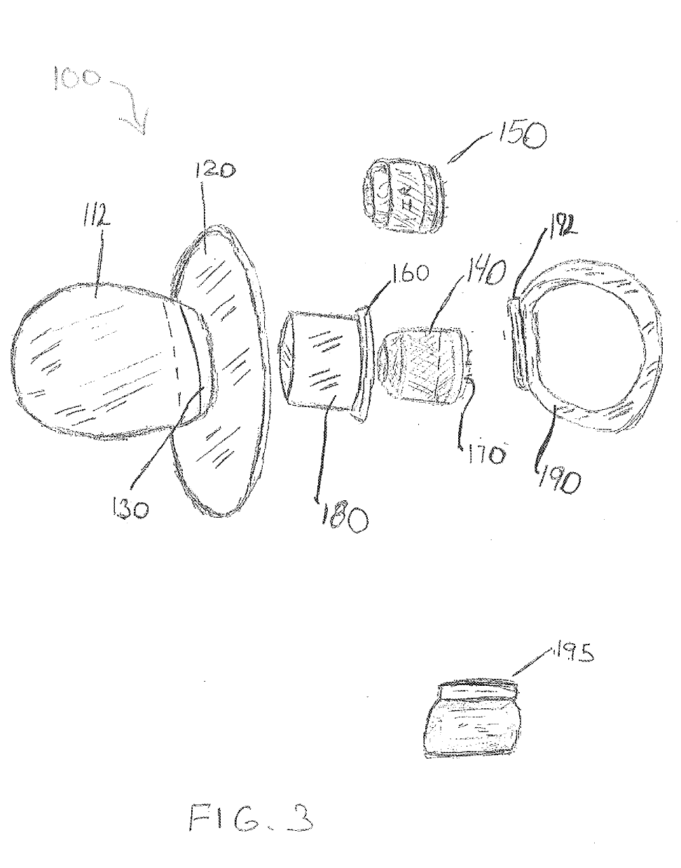

With reference to FIGS. 1 and 2, the interior cavity 16 of the nipple is designed and dimensioned to receive the camera 40 therein. The size of the interior cavity 16 is dependent on the size of the nipple 12. For larger nipples 12, the interior cavity 16 will be larger. For smaller nipples 12, the interior cavity will be smaller. Pacifiers are often sized to fit most infants within a particular age group. For example, small pacifiers may be dimensioned for infants less than six months old; medium pacifiers may be dimensioned for infants and toddlers between six and eighteen months old; large pacifiers may be dimensioned for toddlers between eighteen months and three years old; x-large pacifiers may be dimensioned for children ages three and up; and some pacifiers are even sized to fit the mouth of an adult. Accordingly, the oral examination device 10 disclosed herein may be provided in any of various sizes and the nipple 12 may be dimensioned accordingly in order to properly fit differently sized patients from newborn babies (or even fetuses) to adults. Each differently sized nipple 12 is designed and dimensioned to allow a patient to suck on the nipple 12 such that the nipple 12 fills a significant portion the mouth of the patient during the sucking action, thereby exposing much of the mouth to examination through the nipple 12. In at least one embodiment, the nipple 12 is designed to extend from the lips and teeth to the soft palate of the patient.

The nipple 12 is connected to the shield 20 at the proximal base of the nipple 12. As illustrated in FIGS. 1 and 2, the nipple 12 is provided on a front side of the shield 20, and an opening 22 to the interior cavity 16 is provided on the opposite back side of the shield 20. The opening 22 to the interior cavity 16 is of sufficient size to allow the camera 40 to pass there-through from the back side of the shield 20 in a direction as noted by projection line 97 of FIG. 1.

The nipple 12 is durable and may be used repeatedly with different patients. In such embodiments, the nipple may be permanently fixed to one or more component of the oral examination device 10 (e.g., permanently fixed tot the shield 20, basket 80, or other components). However, in at least one embodiment, the nipple is releasable from the oral examination device 10 and therefore disposable and designed to be replaced with each subsequent use of the oral examination device 10.

With continued reference to FIGS. 1 and 2, the shield 20 is designed and dimensioned in the shape of any of various pacifier guards. Accordingly, the shield may be concave, convex, or substantially planar, with a circular, oval, or other shaped perimeter. The shield 20 is generally sufficiently sized such that it will not easily fit within the mouth of the patient, depending on the size of the oral examination device 10, and will protect the patient from swallowing or chocking on the oral examination device 10. Accordingly, the shield 20 is designed to rest against the outside of the lips of the patient when the nipple 12 is inserted into the mouth of the patient. In at least one embodiment, the shield 20 is made from a stiffer form of phthalates, BPA, lead, and PVC-free silicone. Accordingly, the shield 20 is substantially stiffer and harder than the nipple 12, and the shore durometer of the shield 20 is significantly greater than that of the nipple. However, in at least one embodiment, the shield 20 may be formed of the same material as the nipple 12.

The camera protector 30 is positioned on the interior of the nipple 12 at the opening 22 to the interior cavity and extends slightly inwardly at the proximal base of the nipple 12. The camera protector 30 is generally a ring-like member that defines an exterior and interior and prevents collapse of the outer membrane of the nipple 12 at the proximal base of the nipple 12. The camera protector has a sufficient stiffness and rigidity to prevent collapse of the nipple 12 at the proximal base of the nipple even if a patient bites down on the proximal base of the nipple. Therefore, the camera protector 30 is made from a stiffer form of phthalates, BPA, lead, and PVC-free silicone than the nipple 12. The camera protector 30 is configured to receive and retain the basket 80 and camera 40 therein. Accordingly, the camera protector 30 also serves as a mount for the camera 40 on the oral examination device 10.

The camera 40 may be any of various types of cameras as will be recognized by those of ordinary skill in the art. For example, the camera may be a digital video camera, still photo camera, X-ray camera/device, infrared camera, camera with back-illuminated sensor, etc.; accordingly, the camera 40 may be configured to record any of various different types of images, such as black and white images, color images, digital images, analog images, etc. The camera 40 is typically a miniature camera configured to fit within the interior of the nipple 12, the camera protector 30 and the basket 80. Any of various kinds of micro cameras may be used. Accordingly, the camera is designed and dimensioned such that it, along with the nipple 12, may be easily received within the mouth of a human. For example, in at least one embodiment, the camera 40 has a diameter of less than an inch. A lens configured to allow light (including, for example, visible, infrared, ultraviolet, X-ray, or gamma radiation) to pass there-through is positioned on one end of the camera. Electrical connections are provided on the opposite side of the camera 40.

In at least some embodiments, the camera 40 includes an integral light source built into the camera. Alternatively, a light source may be provided as a separate component from the camera 40, and mounted to any of various locations that may be appropriate for the light source, including locations within the nipple 12 (e.g., secured to the basket 80), or outside of the nipple 12 (e.g. mounted to a base 92) of the handle. In the embodiment of FIG. 1, the sensor 50 may be considered to include the light source or the sensor itself may be the light source. Accordingly, all of the locations discussed herein for placement of the sensor will be understood to further include the possibility of placement of the light source. Typically, the light source is designed to direct light into the mouth of the patient in a manner that facilitates image capture by the camera 40. The light source may be provided in any of various forms, such as an LED, infrared light, or any of various other light sources.

When the oral examination device 10 is assembled, the camera 40 is positioned within the basket 80 on the interior of the nipple 12 and the lens is directed to outwardly from the interior cavity 16 through the one or more transparent areas of the nipple 12 (e.g., the tip 18). In this manner, the camera 40 is configured to receive light and obtain associated images through the one or more transparent areas on the nipple 12. Accordingly, medical and dental professionals will be able to view and take pictures of various areas of the patient's mouth such as the palatopharyngeal arch, palatopharyngeus muscles, oropharynx and any of various other areas of the mouth or throat. As explained in further detail below, the camera may be configured as a wired or wireless camera.

At least one sensor 50 may be positioned on the oral examination device 10. The sensor 50 may be positioned within the interior of the nipple 12 or on the rear side of the shield 20. In at least one embodiment, the sensor 50 is a separate component from the camera and configured to detect or measure at least one physiological parameter or condition of the patient such as temperature, oxygen levels, or the existence of infection. Accordingly, the sensor may be, for example, a thermometer or an oxygen sensor. In at least some embodiments, the sensor 50 is directly associated with the camera in some manner. For example, the sensor 50 may be an image sensor configured to detect image size, resolution, low-light, depth of field, dynamic range, etc. Various configurations for the image sensor are possible such as a front-illuminated sensor or a back-illuminated sensor. In embodiments wherein the sensor 50 is associated with the camera 40, the sensor 50 will typically be located in the nipple 12 along with the camera 40. In embodiments wherein the sensor 50 is separate from the camera 40, the sensor 50 may be positioned outside of the nipple 12 or within the nipple 12, depending on the type of sensor and desired configuration. When the sensor 50 is positioned within the nipple 12, the sensor 50 may advantageously be mounted on the camera itself 40 or on the basket 80. In other embodiments when the sensor 50 is positioned outside of the nipple 12, the sensor 50 may be mounted on the shield 20, the camera protector 30, or any of various other locations that may be appropriate for the sensor.

One or more sanitary gaskets 60 are coupled to the shield 20 and/or the basket 80. The sanitary gasket 60 includes flanges and may be compression controlled. The sanitary gasket 60 may be comprised of any of various materials as will be recognized by those of ordinary skill in the art, including materials approved: USP Class VI, FDA and NSF. The sanitary gaskets 60 seal the space between the basket 80 and the shield 20, and thereby prevent liquids from the mouth of the patient from inadvertently entering the interior cavity 16 of the nipple 12. In at least one embodiment, the gaskets 60 further facilitate coupling of the camera 40, basket 80, and camera protector 30 by allowing the components to easily snap together or by providing a wedge between the basket 80 and the camera guard 30. In at least one embodiment, the sanitary gaskets 60 are disposable and designed to be replaced with each subsequent use of the oral examination device.

In the embodiment of FIG. 1, a wireless connector 70 is connected to the rear side of the camera 40 and is configured to transmit images from the camera 40 to a remote processor, such as a processor in a remote desktop computer, laptop computer, tablet computer, smart phone, or personal electronic device. The wireless connector 70 may include any of various connectors that are configured to electronically connect to the camera 40 and include wireless transmitters configured to transmit data and images received from the camera 40, as will be recognized by those of ordinary skill in the art. In at least one alternative embodiment, the wireless connector 70 is replaced with a wired connector that allows data and images from the camera to be sent to a processor over a physical cable. Together, the camera protector 30 and wireless connector 70 allow the oral examination device to be used without the interference of wires or cords while also preventing damage to the camera during examinations.

The basket 80 is connected to the camera protector 30. The basket 80 is a cylindrical component that is configured to receive and pass the camera 40, thus allowing the camera 40 to extend into the interior of the nipple 12. The basket 80 is short enough to extend into the nipple 12 without reaching the tip 18, but is long enough such that most or the entire length of the camera 40 within the nipple 12 is surrounded by the basket 80. The wireless connector 70 may also be received within the basket 80, with at least a portion of the wireless connector 70 retained within the basket 80. The basket 80 may also be configured to receive and retain any of various additional electronic components for the oral examination device 10, such as electronic components for the sensor 50 and any of various wireless connections made from the oral examination device 10.

The outer surface of the proximal end of the basket 80 is snugly received by the camera protector 30 and/or the sanitary gaskets 60. Accordingly, the proximal end of the basket 80 is supported, but the distal end of the basket 80 extends into the interior cavity 16 of the nipple 12 in a cantilever manner. The inner surface of the basket 80 is configured to engage clips 82 that retain the camera 40 and extend between the camera and the handle 90.

In at least one embodiment, the basket is made from a stiffer form of phthalates, BPA, lead, and PVC-free silicone than the nipple 12 and has a significantly greater shore durometer than the nipple 12. Accordingly, in addition to serving as a mount for the camera 40, the basket 80 also protects the camera 40 within the nipple, preventing damage to the camera 40 should a patient clamp down on the nipple 12. In at least one embodiment, the camera 40 is moveable relative to the basket 80 and/or the camera protector 30. However, in other embodiments, the camera 40 is stationary relative to the basket 80 and the camera protector 30.

As noted previously, the handle 90 is connected to the camera 40 via the clips 82. In the embodiment of FIG. 1, the inner surface of the clips 82 snugly engage the camera 40 and the outer surface of the clips 82 snugly engage the inner surface of basket 80. In this manner, the clips 82 mount the camera to the basket 80 and prevent the camera 40 from being released from the oral examination device 10. In at least one embodiment, the clips are comprised of a relatively hard and rigid material defined by a relatively high shore durometer. Because the clips 82 are stiff, movement of the handle 90 on the exterior of the nipple 12 is translated to the camera 40 within the interior cavity 16 of the nipple 12. However, in at least one alternative embodiment, the clips 82 may be comprised of a rubber or elastomer.

The handle 90 is a member that allows a medical or dental professional to grasp the handle 90 with his or her fingers and manipulate the oral examination device 10 for insertion in the mouth of the patient. In at least one embodiment, the handle 90 and camera 40 are configured to move at least somewhat relative to the basket 80 and nipple 12, thus allowing the professional to manipulate the camera 40 within the mouth of the patient. For example, depending on the hardness and elasticity of the sanitary gaskets 60, a torque applied to the handle 90 may cause the sanitary gaskets 60 to slightly deform, and thus allow the professional to slightly adjust the direction the camera 40 is oriented within the interior cavity 16 of the nipple 12. Moreover, the handle 90 allows the professional to adjust the position of the nipple 12 and the associated camera 40 within the mouth of the patient, and thereby orient the camera in a more desirable position for image capture.

The handle 90 may be a ring-like member as shown in FIG, 1, a post, or any of various other configurations designed to be held and manipulated by the human hand. The handle includes a base 92 that designed to be directly coupled to either the interior or exterior surface of the clips 82. The handle 90 may be made from a relatively form of phthalates, BPA, lead, and PVC-free silicone.

While the oral examination device 100 is shown in an exploded view in FIG. 1, it will be recognized that the components of the camera are designed to be coupled together along projection line 97. Accordingly, when the oral examination device 100 is assembled, the base 92 of the handle 90 is positioned in close proximity to the camera protector 30 and the shield 20, with the basket 80 and camera 40 positioned substantially or completely within the interior cavity 16 of the nipple 12. It will be recognized that the nipple 12 of the oral examination device 10 of FIG. 1 is configured to fit easily within the mouth of the infant or toddler and alleviate discomfort and trauma for infant. However, in at least some embodiments, the oral examination device is configured to fit within the mouth of an adult. The oral examination device 10 simplifies medical examinations and testing for patients, including infant patients in the NICU or Nursery department in the hospital, providing for the early detection of problems such as swallowing, swollen glands, pallet deformities etc.

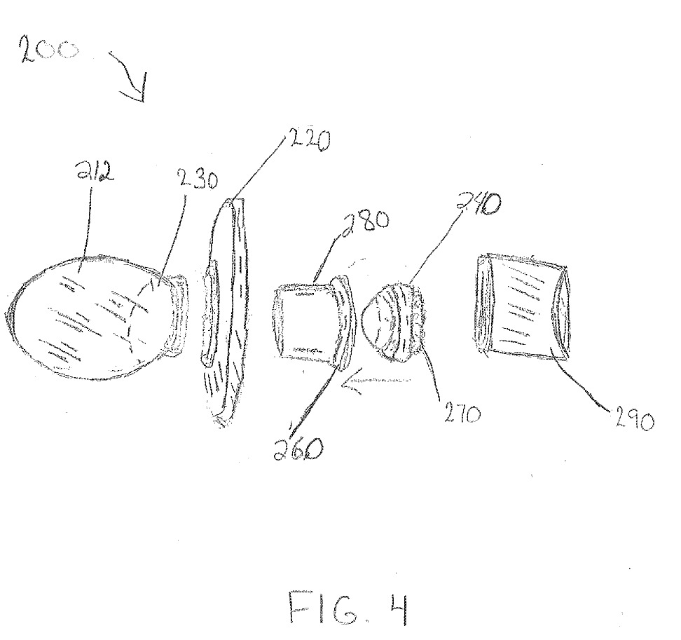

With reference now to FIG. 3, in at least one alternative embodiment, an oral examination device 100 includes a nipple 112, a shield 120, a camera protector 130, a camera 140, a sensor 150, a sanitary gasket 160, a wireless connector 170, a basket 180, a handle 190, and a consumable product 195. The nipple 112, shield 120, camera protector 130, camera 140, sensor 150, sanitary gasket 160, wireless connection 170, basket 180, and handle 190 are similar to the related components of FIG. 1, buy may be provided in different forms or configurations. For example, the sanitary gasket 160 may be larger than the opening to the interior cavity of the nipple 112, and therefore designed to abut the shield 120 on the outside of the opening 122. As another example, the nipple 112 may be a cost-effective disposable nipple that is removable from the shield or basket 180. As yet another example, the sensor 150 may be an infrared sensor that is interchangeable with the video camera 140, the infrared sensor allowing the oral examination device 100 to more easily check for infection in the mouth or throat of the patient. Accordingly, it will be recognized that the term “camera” as used herein is not limited to video or still picture cameras, but includes all devices that detect light in some form.

In addition to the above, in the embodiment of FIG. 3, a consumable product 195 may be used to coat the nipple 112 prior to insertion into the mouth of the patient. The consumable product 195 may be a flavored, liquid, gel or paste that the nipple 112 is dipped in and then offered to the patient. The consumable product 195 encourages the infant, child or other patient to suck on the nipple 112 when the oral examination device 100 is in use.

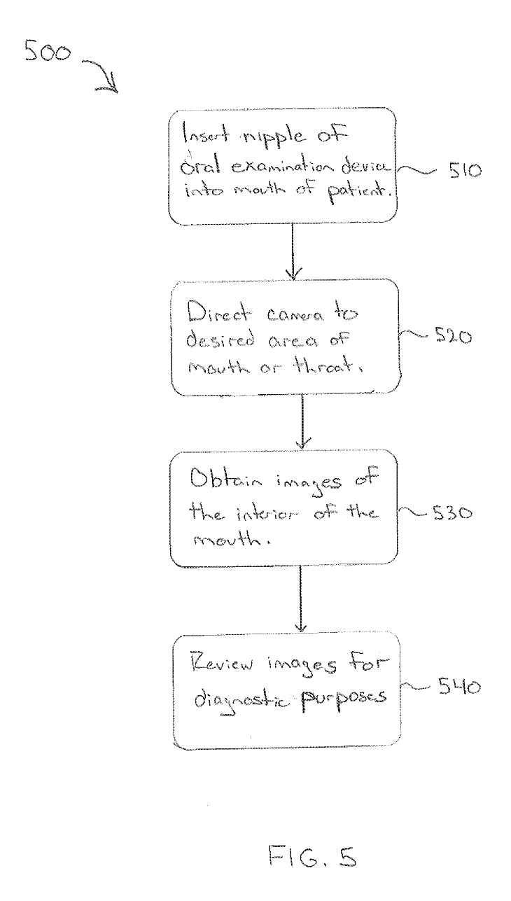

With reference now to FIG. 4, in at least one embodiment, an oral examination device 200 is configured to simplify oral examinations, X-rays and scanning for dentures allowing more views of the patient's teeth, regardless of whether the patient is a child or an adult. Accordingly, it will be recognized that the oral examination device 200 may be provided in different forms and size configurations depending on the intended patient and use for the oral examination device. In the embodiment of FIG. 4, the oral examination device 200 includes a modified adult-sized nipple 212, a shield 220, a camera protector 230, a camera and scanner 240, one or more sanitary gaskets 260 and associated flanges, a wireless connection 270 associated with the camera and scanner 240, a basket 280, and a handle 290.

With reference now to FIG. 5, in operation, a block diagram of a method 500 for conducting an oral examination is shown. The method 500 begins with step 510, when the nipple 12 of the oral examination device 10 is inserted into the mouth of the patient. Infants, in particular, will naturally receive the device within the mouth. However, if coaxing is needed, the nipple 12 may be dipped in a liquid or gel that provides a good taste to the mouth of the patient. As noted in step 520, after the nipple 12 of the oral examination device 10 is in place in the patient's mouth, the camera 40 is directed to a desired area of the mouth or throat of the patient. Next, in step 530, the camera 40 is used to obtain images of the interior of the mouth of the patient, such as images showing views of the teeth and throat. As noted previously, the device 10 may be manipulated to obtain any of various desired images on the interior of the mouth, allowing more views of the patient's teeth, mouth and throat than would be available without the oral examination device 10. Next in step 540, the images obtained with the oral examination device 10 are used for any of various diagnostic purposes, including medical and dental examinations, X-rays, and scanning for denture fitting. The oral examination device 10 alleviates discomfort and trauma during oral examinations and X-rays and produces improved images over previous oral examination devices.

While the oral examination device 10 has been described herein in association with various exemplary embodiments and for various exemplary uses, it will be recognized that the oral examination device 10 may be used for other purposes. For example, in at least one embodiment, the oral examination device is configured for use with any of various species of animals.

The foregoing detailed description of one or more exemplary embodiments of the oral examination has been presented herein by way of example only and not limitation. It will be recognized that there are advantages to certain individual features and functions described herein that may be obtained without incorporating other features and functions described herein. Moreover, it will be recognized that various alternatives, modifications, variations, or improvements of the above-disclosed exemplary embodiments and other features and functions, or alternatives thereof, may be desirably combined into many other different embodiments, systems or applications. Presently unforeseen or unanticipated alternatives, modifications, variations, or improvements therein may be subsequently made by those skilled in the art which are also intended to be encompassed by the appended claims. Therefore, the spirit and scope of the present disclosure and any eventually appended claims should not be limited to the description of the exemplary embodiments contained herein.

What is claimed is:

1. An oral examination device, the device comprising:
a lip shield defining a front side and back side;
a nipple extending from a front side of lip shield, the nipple defining an interior cavity with an opening to the interior cavity provided from a rear side of the lip shield; and
a camera positioned in the interior cavity of the nipple.

2. The device of claim 1 wherein the nipple is comprised of at least one substantially transparent area and the camera is directed outwardly from the interior cavity through the transparent area and configured to obtain images through the transparent area.

3. The device of claim 1 further comprising a camera protector positioned inside of the interior cavity of the nipple.

4. The device of claim 3 wherein the camera protector is provided by a ring-like member configured to support the nipple and prevent collapse of the nipple onto the camera when a force directed toward the interior cavity is applied to an exterior of the nipple.

5. The device of claim 4 wherein the camera protector is connected to a cylindrical basket, the cylindrical basket designed and dimensioned to receive the camera.

6. The device of claim 1 wherein the camera is a video camera, still photo camera, an X-ray device, or an infrared sensor.

7. The device of claim 6 wherein the camera is a wireless camera.

8. The device of claim 1 further comprising at least one sensor supported by the lip shield, the at least one sensor configured to measure at least one physiological parameter.

9. The device of claim 1 wherein the at least one sensor is at least partially positioned in the interior cavity of the nipple.

10. The device of claim 1 further comprising a handle connected to the camera.

11. A method of conducting an oral examination on a patient, the method comprising:
inserting a nipple into a mouth of the patient, the nipple including an interior cavity with a camera positioned in the interior cavity; and
obtaining images with the camera.

12. The method of claim 11 wherein the nipple includes at least one transparent area.

13. The method of claim 11 further comprising manipulating the camera by moving a handle attached to the camera.

14. The method of claim 11 wherein the camera is a wireless camera, the method further comprising wirelessly transmitting the images with the wireless camera.

15. The method of claim 11 wherein the patient is an infant and the nipple is designed and dimensioned to be received in the mouth of the infant.

16. The method of claim 11 further comprising coating the nipple with a consumable product prior to inserting the nipple into the mouth of the patient.

17. The method of claim 11 further comprising detecting at least one physiological parameter of the patient using a sensor coupled to the nipple.

18. An oral examination device comprising:
a flexible nipple designed and dimensioned to be received in a mouth of a patient, the flexible nipple defining an interior cavity; and
a camera positioned in the interior cavity of the nipple.

19. The oral examination device of claim 18 wherein the flexible nipple is coated with a consumable product.

20. The oral examination device of claim 19 wherein the patient is an adult human.
